(19) United States
(12) Patent Application Publication
(10) Pub. No.: US 2026/0232747 A1
(43) Pub. Date: Aug. 13, 2026

(54) Composition of Ginseng, Cistanche deserticola, and Polygonatum sibiricum with Ability to Improve Erectile Dysfunction Caused by Aging, and Preparation Method Therefor

(71) Applicant: Jiangnan University, Wuxi (CN)

(72) Inventors: Lianfu ZHANG, Wuxi (CN); Jialing CAO, Wuxi (CN); Xiaoyan XU, Wuxi (CN)

(21) Appl. No.: 19/637,291

(22) Filed: Apr. 2, 2026

Related U.S. Application Data
(63) Continuation of application No. PCT/CN2025/102892, filed on Jun. 24, 2025.

(30) Foreign Application Priority Data
Feb. 5, 2025 (CN) 2025101300251

(51) Int. Cl. A61K 36/258; A61K 36/64; A61K 36/8969; A61P 15/10

(52) U.S. Cl. CPC A61K 36/258; A61K 36/64; A61K 36/8969; A61P 15/10; A61K 2236/11; A61K 2236/17

(57) ABSTRACT

Disclosed are a composition of ginseng, Cistanche deserticola, and Polygonatum sibiricum with an ability to improve erectile dysfunction caused by aging, and a preparation method therefor. The method includes: (1) adjusting a moisture content of raw materials of a processed product to 60%-80% and sealing the raw materials; (2) insulating sealed raw materials; (3) taking out insulated raw materials and drying the raw materials until a moisture content thereof decreases to 15%-40%; (4) sealing and insulating the raw materials from step (3); and (5) taking out the raw materials from step (4) to obtain the processed product. The present disclosure improves contents of glucose and maltose in the raw materials of the ginseng, the Cistanche deserticola, and the Polygonatum sibiricum by pretreatment, and prepares a functional food ingredient. A functional food ingredient has been experimentally verified to have a significant effect on improving erectile dysfunction caused by aging.

### TECHNICAL FIELD

The present disclosure relates to the field of food processing, and in particular to a composition of ginseng, Cistanche deserticola, and Polygonatum sibiricum with an ability to improve erectile dysfunction caused by aging, and a preparation method therefor.

### BACKGROUND

Ginseng grown for five years or less and Polygonatum sibiricum (Huangjing) are both medicinal and edible raw materials, and their traditional processing methods involve one or more cycles of steaming—sun drying treatments (Polygonatum sibiricum traditionally undergoes nine cycles of steaming—sun drying treatments). Processed products obtained from ginseng are called red ginseng, processed Polygonatum sibiricum are called Xianren Yuliang, both of them are believed to have effects of anti-aging and promoting male reproductive health. The medicinal and edible raw material Cistanche deserticola is believed to have effects of improving male health and softening blood vessels.

Traditionally, beneficial health effects of red ginseng are believed to be a presence of low polarity saponins such as Rg2, Rh1, Rg3, and ginseng polysaccharides; a function of the processed Polygonatum sibiricum is usually attributed to polysaccharides from Polygonatum sibiricum, flavonoids, and steroidal saponins; and an improvement effect of Cistanche deserticola on male health is believed to be due to an action of components such as phenylethanoid glycosides, iridoids, and lignans. However, what is inconsistent with these traditional understandings is that during processing of the medicinal and edible raw materials (e.g., red ginseng processed from ginseng grown for five years or less, processed Polygonatum sibiricum grown for five years or less, and traditionally dried Cistanche deserticola grown for five years or less), there is often a certain degree of degradation of polysaccharides, partial conversion of saponins into glycosides, and loss of thermosensitive components of flavonoids. If these components indeed take effect, an efficacy of the processed raw materials may be weakened due to a decrease in a content of these bioactive components. Therefore, we firmly believe that during the processing of these medicinal and edible raw materials, there is a high possibility that new components with strong physiological activity may be formed due to reactions between components that originally exist and have average functional activity in the raw materials.

Based on the above reasons, a systematic analysis of a processing technology/experience of traditional medicinal and edible raw materials is conducted to confirm newly formed components and mechanisms of action thereof in the processed raw materials, and through precise control of a processing process, a content of these new components is increased, and beneficial health effects thereof are strengthened, which is of great significance for improving scientific nature of the processing technology of the medicinal and edible raw materials, standardizing a production process, and achieving controllable product quality and safeguarding health rights and interests of consumers.

### SUMMARY

### Technical Problem

At present, there is a lack of systematic analysis of a processing process of traditional medicinal and edible raw materials, and there is a lack of in-depth research on changes in a composition of raw materials and functions of processed products during the processing process. Therefore, the processing process of traditional medicinal and edible raw materials, such as ginseng, Cistanche deserticola, and Polygonatum sibiricum, is to be explored in order to understand the changes in the composition of the raw materials and optimize a processing technology thereof.

### Technical Content

The study of the present disclosure found that contents of glucose and maltose in ginseng, Cistanche deserticola, and Polygonatum sibiricum grown for five years or less can be increased through pretreatment of raw materials. Arginine is the dominant amino acid in all the three raw materials after pretreatment, accounting for more than 50% of a total free amino acid content. Traditional processing technologies usually involve a certain period of thermal processing stage at a low system moisture (5%-25%) and a high temperature (95° C.-110° C.), which is conducive to Maillard initial reaction between free amino acids and reducing sugars, forming stable and potentially physiologically active Amadori compounds (ACs). During this treatment stage, arginine in the ginseng, Polygonatum sibiricum, and Cistanche deserticola grown for five years or less may undergo Maillard initial reaction with the maltose and glucose, forming arginyl-fructosyl-glucose (AFG) and fructosyl-arginine (FruArg). Especially, when a mass ratio of AFG to FruArg in a product system is 5:1 to 1:3, and a sum of the two accounts for 2.5%-8% of the product mass, the product has a significant effect on improving erectile dysfunction caused by aging.

A first objective of the present disclosure is to provide a composition for alleviating male sexual dysfunction caused by aging, and the composition includes arginyl-fructosyl-glucose (AFG) and fructosyl-arginine (FruArg).

Further, a mass ratio of AFG to FruArg in the composition is 8:1 to 1:3.

Further, the mass ratio of AFG to FruArg in the composition is 6:1 to 1:3.

Preferably, the mass ratio of AFG to FruArg in the composition is 5:1 to 1:3.

Further, the AFG may be prepared according to the following method: adding maltose and arginine to water, adjusting a pH of a solution to neutral, starting heating for reaction, and then performing purification and separation to obtain the AFG.

Further, a molar ratio of the maltose to the arginine is (0.5-1.5):1.

Further, a pH value of the solution is 6.5-7.5.

Further, the heating for reaction is performed at 80° C.-90° C. for 0.5-2 h.

Further, column purification or gas chromatography purification may be performed to achieve the purification; and when column purification is performed, Dowex 50WX4 ion exchange resin may be used.

Further, the FruArg may be prepared according to the following method: adding glucose and arginine to water, adjusting a pH of a solution to neutral, starting heating for reaction, and then performing purification and separation to obtain the FruArg.

Further, a molar ratio of the glucose to the arginine is (0.5-1.5):1.

Further, a pH value of the solution is 6.5-7.5.

Further, the heating for reaction is performed at 80° C.-90° C. for 0.5-2 h.

Further, column purification or gas chromatography purification may be performed to achieve the purification; and when column purification is performed, Dowex 50WX4 ion exchange resin may be used.

A second objective of the present disclosure is to provide an application of the aforementioned composition for alleviating male sexual dysfunction caused by aging in drug preparation.

A third objective of the present disclosure is to provide a processed product for alleviating male sexual dysfunction caused by aging, where a mass ratio of AFG to FruArg in the processed product is 8:1 to 1:3.

Further, a mass ratio of AFG to FruArg in the processed product is 6:1 to 1:3.

Preferably, the mass ratio of AFG to FruArg in the processed product is 5:1 to 1:3.

Further, the processed product includes one or more of red ginseng, Cistanche deserticola, and Polygonatum sibiricum.

A fourth objective of the present disclosure is to provide a method for preparing a processed product with a high AFG and FruArg content and an ability to alleviate male sexual dysfunction caused by aging, and the method includes the following steps:
(1) adjusting a moisture content of raw materials of the processed product to 60%-80% and sealing the raw materials;
(2) insulating sealed raw materials of the processed product;
(3) taking out insulated raw materials of the processed product and drying the raw materials until a moisture content thereof decreases to 15%-40%;
(4) sealing and insulating the raw materials of the processed product from step (3); and
(5) taking out the raw materials of the processed product from step (4) and drying the raw materials until a moisture content thereof is 10% or lower, to obtain the processed product.

Further, the raw materials of the processed product in step (1) are one or more of ginseng, Cistanche deserticola, and Polygonatum sibiricum.

Further, the ginseng, the Cistanche deserticola, and the Polygonatum sibiricum are grown for five years or less.

Further, the raw materials of the processed product may be intact raw materials, cut raw materials, pulverized raw materials, or pulped raw materials.

Further, when the raw materials of the processed product are a mixture of the ginseng and the Polygonatum sibiricum, a mass ratio of the ginseng to the Polygonatum sibiricum is (0.5-1.5):1.

Further, when the raw materials of the processed product are a mixture of the ginseng and the Cistanche deserticola, a mass ratio of the ginseng to the Cistanche deserticola is (1.5-2.5):1.

Further, when the raw materials of the processed product are a mixture of the Polygonatum sibiricum and the Cistanche deserticola, a mass ratio of the Polygonatum sibiricum to the Cistanche deserticola is (0.5-1.5):2.

Further, when the raw materials of the processed product are a mixture of the ginseng, the Polygonatum sibiricum, and the Cistanche deserticola, a mass ratio of the ginseng to the Polygonatum sibiricum to the Cistanche deserticola is (0.5-1.5):(0.5-1.5):1.

Further, a process of adjusting the moisture content in step (1) is specifically: placing the raw materials of the processed product at 50° C.-60° C. to remove moisture.

Further, the moisture content in step (1) is adjusted to 65%-80%.

Further, sealing and insulation in step (2) are performed at 50° C.-60° C. for 48-72 h.

Further, drying in step (3) includes high-temperature drying, natural air drying, or drying under reduced pressure.

Further, the high-temperature drying is performed at 90° C.-105° C.

Further, the moisture content in step (3) is reduced to 15%-30%.

Further, the raw materials of the processed product from step (3) in step (4) may be any one of the ginseng, the Cistanche deserticola, and the Polygonatum sibiricum treated in steps (1) to (3), or a combination of any two or three of the ginseng, the Cistanche deserticola, and the Polygonatum sibiricum treated separately in steps (1) to (3), or a combination of any two or three of the ginseng, the Cistanche deserticola, and the Polygonatum sibiricum treated together in steps (1) to (3).

Further, sealing and insulation in step (4) are performed at 90° C.-110° C. for 30-150 min.

Further, drying in step (5) includes high-temperature drying, natural air drying, or drying under reduced pressure.

Further, the high-temperature drying is performed at 90° C.-105° C.

Further, the moisture content after drying in step (5) is 8%-10%.

### Beneficial Effects of the Present Disclosure:

The present disclosure improves contents of glucose and maltose in the raw materials of the ginseng, the Cistanche deserticola, and the Polygonatum sibiricum by pretreatment, and adjusts a composition and a proportion of three raw materials based on contents of glucose, maltose, and arginine in the three raw materials to obtain a combined material with a dry basis content of glucose or maltose of not less than 2%, a dry basis content of a sum of glucose and maltose of 5% or more, an arginine content of not less than 1.5% (dry basis), and a proportion of arginine to total free amino acids of not less than 50%. The combined material may be prepared into a functional food ingredient with a sum of the AFG content and FruArg content of 2.5%-8% and a mass ratio of the AFG to the FruArg of 5:1 to 1:3 through technologies such as moisture adjustment and sealed insulation. The functional food ingredient has been experimentally verified to have a significant effect on improving erectile dysfunction caused by aging.

### BRIEF DESCRIPTION OF FIGURES

FIG. 1 shows chemical structural formulas of arginyl-fructosyl-glucose (AFG) and fructosyl-arginine (FruArg).

FIG. 2 shows mass spectra (ESI+mode) of self-synthesized AFG and FruArg standard samples.

FIG. 3 shows chromatograms of self-synthesized AFG and FruArg standard samples obtained by a high performance liquid chromatography tandem evaporative light scattering detector.

FIG. 4 shows contents of glucose and maltose in raw materials before and after pretreatment in Example 2.

FIG. 5 shows UPLC-MS/MS total ion chromatograms of AFG and FruArg in a sample processed in Example 2.

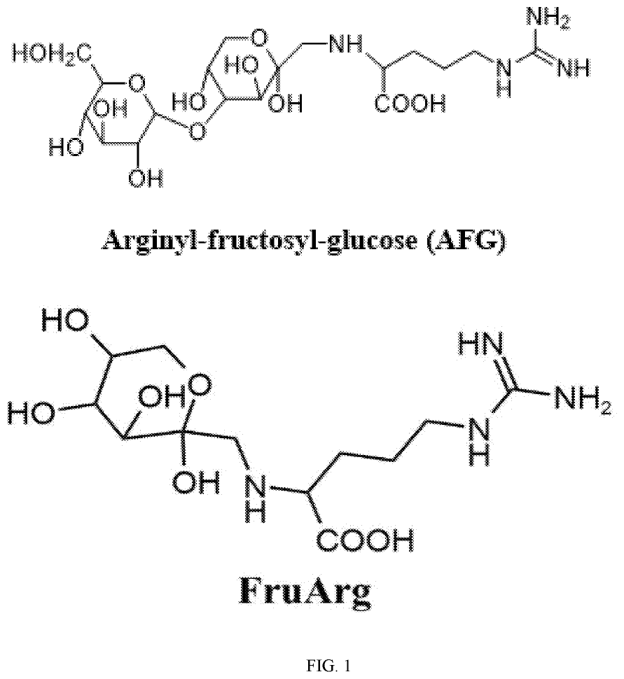

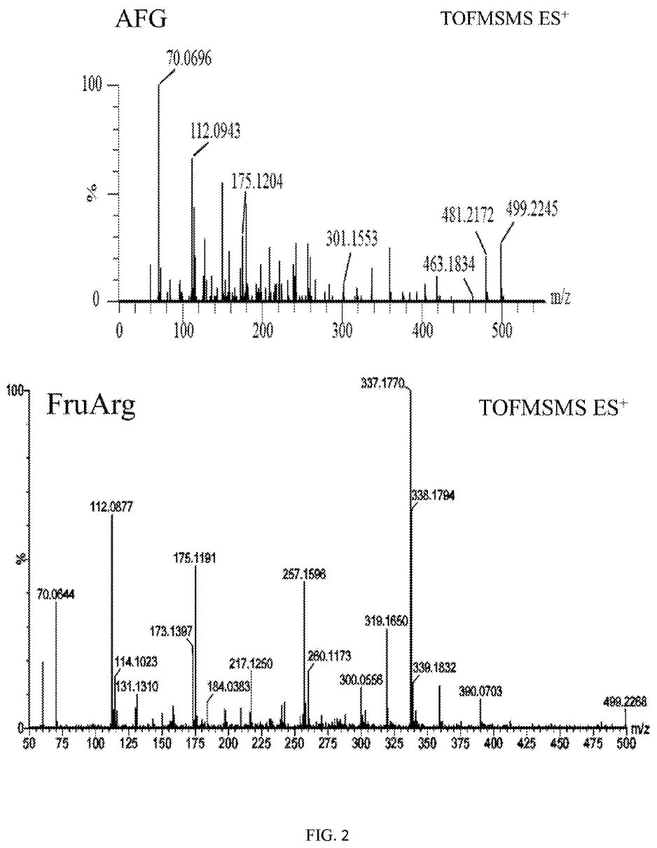

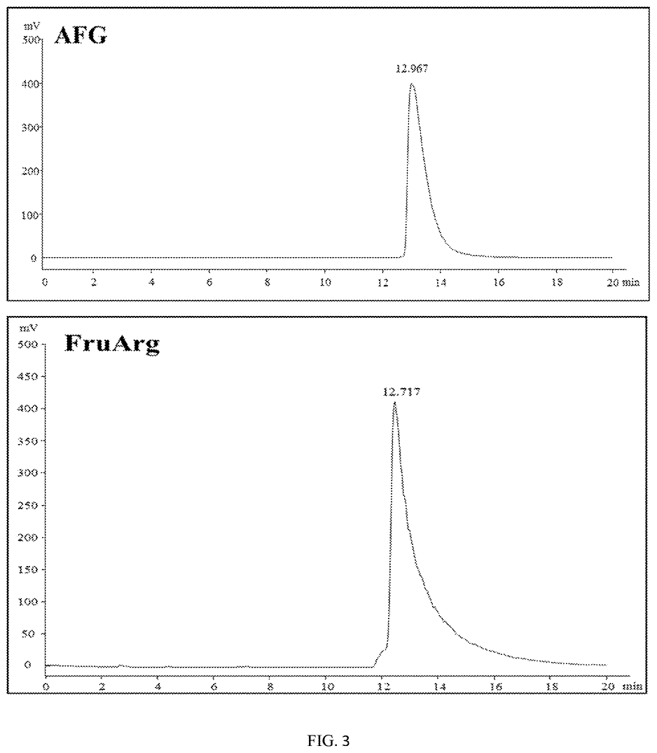

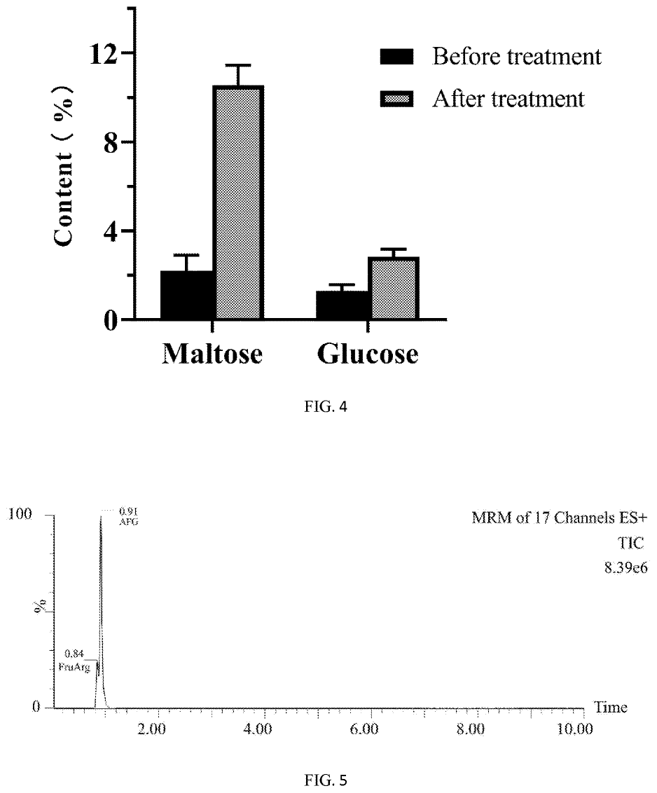

### DETAILED DESCRIPTION

In order to make the foregoing objectives, features, and advantages of the present disclosure clearer and more comprehensible, the specific implementations of the present disclosure are described in detail below with reference to the examples in the description.

In the following description, many specific details are set forth in order to facilitate full understanding of the present disclosure, but the present disclosure can also be implemented in other ways other than those described herein. Those skilled in the art can make similar expansion without departing from the connotation of the present disclosure. Therefore, the present disclosure is not limited by the specific examples disclosed below.

In addition, “an example” or “examples” used herein refers to particular features, structures, or characteristics that are included in at least one implementation of the present disclosure. “In an example” appearing in different places in this description is not intended to refer to the same example, nor is it a separate or selective example that is mutually exclusive with other examples.

### Source of Raw Materials

D-galactose was purchased from Beijing Solarbio Technology Co., Ltd; Tadalafil was purchased from Menarini (China) Co., Ltd; ginseng was purchased from Fusong County, Jilin Province; and Polygonatum sibiricum was purchased from Nanchang City, Jiangxi Province.

Arginyl-fructosyl-glucose (AFG) and fructosyl-arginine (FruArg) are both self-made. A specific process for preparing the AFG is as follows: Maltose and arginine are dissolved in water at a molar ratio of 1:1, a pH of a solution is adjusted to 7.4, the solution is heated for reaction at 90° C. for 1 h in a rotary evaporator, and then Dowex 50WX4 ion exchange resin (hydrogen type) is used to separate AFG from a reaction mixture. A purity of an obtained AFG tested by a high-performance liquid chromatography tandem evaporative light scattering detector is 99.61%. A specific process for preparing the FruArg is as follows: Glucose and arginine are dissolved in water at a molar ratio of 1:1, a pH of a solution is adjusted to 7.4, the solution is heated for reaction at 90° C. for 1 h in a rotary evaporator, and then Dowex 50WX4 ion exchange resin (hydrogen type) is used to separate FruArg from a reaction mixture. A purity of an obtained FruArg tested by the high-performance liquid chromatography tandem evaporative light scattering detector is 98.83%.

### Test Process

Contents of AFG, FruArg, 5-HMF, and acrylamide (AA) in various processed products in the following examples are tested by qualitative and quantitative analysis within a 10-min operation time by an optimized ultra-high performance liquid chromatography tandem mass spectrometry (UPLC-MS/MS) method. A specific process is as follows:
1. Simultaneous qualitative and quantitative analysis method for AFG and FruArg
1.1 Instrument: Ultra-high performance liquid chromatography tandem quadrupole mass spectrometer (UPLC-TQD, Waters Corporation, USA); Chromatographic column: ACQUITY UPLC BEH C18 (100×2.1 mm, 1.7 μm);
1.2 Liquid chromatography conditions: Mobile phase: 100% acetonitrile (phase A) and 0.1% formic acid aqueous solution (v/v) (phase B); Column temperature: 35° C.; Flow rate: 0.3 mL/min; Injection volume: 3 μL; Gradient elution procedure: during 0-5 min, a volume fraction of phase B is changed from 100% to 90%; during 5-6 min, the volume fraction of phase B is changed from 90% to 50%; during 6-7 min, the volume fraction of phase B is changed from 50% to 20%; during 7-9 min, the volume fraction of phase B is maintained at 20%; and during 9-10 min, the volume fraction of phase B is changed from 20% to the initial 100% B; and Total running time: 10 min.
1.3 Mass spectrometry test conditions: An electrospray ionization source is provided and a positive ion mode (ESI+) is selected, Ion source temperature: 120° C.; Desolvation gas temperature: 400° C.; Capillary voltage: 3.0 kV; Detector voltage: 1800 V; and Purity of high-purity nitrogen (N₂): greater than 99.99%. Flow rate of cone gas (N₂): 50 L/h; Flow rate of desolvation gas (N₂): 800 L/h; and Scanning range of mass spectrometry: 50-1000 m/z. A multiple reaction monitoring (MRM) mode is used for precise quantitative analysis, and optimized parameters such as cone voltage, collision energy, and quantitative product ions are shown in Table 1.

TABLE 1
Mass spectrometry parameters of AFG and FruArg

| | Cone voltage | Collision | Parent ions | Quantitative |
|---|---|---|---|---|
| Analyte | (eV) | energy (eV) | (m/z) | ions (m/z) |
| AFG | 40 | 5 | 499.68 | 499.68 |
| FruArg | 30 | 20 | 337.10 | 114.03 |

2. Qualitative and quantitative analysis method of 5-hydroxymethylfurfural (5-HMF) and acrylamide (AA) by UPLC-MS/MS
2.1 Instrument: Ultra-high performance liquid chromatography tandem quadrupole mass spectrometer (UPLC-TQD, Waters Corporation, USA); Chromatographic column: ACQUITY UPLC BEH C18 (100×2.1 mm, 1.7 μm);
2.2 Liquid chromatography conditions: Mobile phase: 100% acetonitrile (phase A) and 0.1% formic acid aqueous solution (v/v) (phase B); Column temperature: 35° C.; Flow rate: 0.3 mL/min; Injection volume: 3 μL; Gradient elution procedure: during 0-5 min, the volume fraction of phase B is changed from 100% to 50%; during 5-10 min, the volume fraction of phase B is changed from 50% to 0%; during 10-12 min, the volume fraction of phase B is changed from 0% to 100%; and during 12-15 min, the volume fraction of phase B is maintained at 100%; and Total running time: 15 min.
2.3 Mass spectrometry test conditions are the same as those in 1.3. The MRM mode is used for precise quantitative analysis, and optimized parameters such as cone voltage, collision energy, and quantitative product ions are shown in Table 2.

TABLE 2
Optimized mass spectrometry parameters of 5-HMF and AA

| | Cone voltage | Collision | Parent ions | Quantitative |
|---|---|---|---|---|
| Analyte | (eV) | energy (eV) | (m/z) | ions (m/z) |
| 5-HMF | 30 | 13 | 126.60 | 109.00 |
| AA | 26 | 13 | 72.1 | 55.49 |

### Example 1: Evaluation of Effect of Composition of AFG and FruArg on Improving Erectile Dysfunction Caused by Aging

Experimental animals: 64 healthy male 8-week-old SPF SD rats.

Experimental grouping: Group C: Blank control group, administered with 0.5% CMC-Na normal saline by gavage; Group D: D-galactose (D-gal) model control group, administered with 0.5% CMC-Na normal saline by gavage; Group P: Tadalafil positive control group, administered with 0.52 mg/kg BW by gavage;

Sample-1 group: The gavage sample is a composition prepared in a mass ratio of AFG:FruArg=5:1. The gavage sample is 40 mg/kg BW, calculated as the sum of AFG and FruArg;

Sample-2 group: The gavage sample is a composition prepared in a mass ratio of AFG:FruArg=1:3. The gavage sample is 40 mg/kg BW, calculated as the sum of AFG and FruArg;

Sample-3 group: The gavage sample is a composition prepared in a mass ratio of AFG:FruArg=2:1. The gavage sample is 40 mg/kg BW, calculated as the sum of AFG and FruArg;

Sample-4 group: The gavage sample is a composition prepared in a mass ratio of AFG:FruArg=10:1. The gavage sample is 40 mg/kg BW, calculated as the sum of AFG and FruArg;

Sample-5 group: The gavage sample is a composition prepared in a mass ratio of AFG:FruArg=1:5. The gavage sample is 40 mg/kg BW, calculated as the sum of AFG and FruArg;

Except Group C, the other groups received intraperitoneal injections of D-gal at a dose of 150 mg/kg BW daily for 9 consecutive weeks. From the 6th week, intraperitoneal injection and gavage administration were performed simultaneously for 4 consecutive weeks.

### Test Results:

### 1) the Effect of the Composition of AFG and FruArg on Aging Body Weight and Penis Weight

TABLE 3
Average body weight and penis weight of rats
in each group during the experimental process

| | Average body weight change (g) | Penis weight (g) |
|---|---|---|
| C | 230.14 ± 10.92a | 0.77 ± 0.07a |
| D | 226.57 ± 9.98a | 0.77 ± 0.05a |
| P | 232.86 ± 10.49a | 0.79 ± 0.06a |
| Sample-1 | 231.41 ± 10.38a | 0.79 ± 0.07a |
| Sample-2 | 230.89 ± 10.78a | 0.79 ± 0.05a |
| Sample-3 | 232.37 ± 11.82a | 0.80 ± 0.06a |
| Sample-4 | 231.65 ± 10.85a | 0.78 ± 0.05a |
| Sample-5 | 229.81 ± 9.65a | 0.79 ± 0.04a |

Note:
“a” represents the same indicator, and different groups have no significant difference (P > 0.05).

As shown in Table 3, the groups of rats have no significant difference in body weight and penis weight (P>0.05), indicating that the composition of AFG and FruArg has no significant regulatory effect on the body weight and penis weight of rats.

### 2) the Effect of the Composition of AFG and FruArg on Aging-Related Marker Genes in the Aging Penis

TABLE 4
Relative expression values of aging-related marker
genes in each experimental group of rats

| | P53 | P21 |
|---|---|---|
| C | 1.00 ± 0.06a | 1.00 ± 0.05a |
| D | 14.55 ± 1.98 c | 10.3 ± 1.92 c |
| P | 2.12 ± 0.47a | 1.26 ± 0.22a |
| Sample-1 | 2.37 ± 0.42a | 1.91 ± 0.41a |
| Sample-2 | 2.45 ± 0.47a | 2.11 ± 0.49a |
| Sample-3 | 2.13 ± 0.41a | 2.03 ± 0.37a |
| Sample-4 | 4.75 ± 0.55 b | 3.76 ± 0.59 b |
| Sample-5 | 5.32 ± 0.83 b | 4.49 ± 0.77 b |

Note:
“a-c” indicates that groups with different letters in the same indicator have significant differences (P < 0.05).

As shown in Table 4, compared with the D-gal model group, the AFG and FruArg composition groups of rats showed a significant decrease in the expression of related marker genes p53 and p21 in the penis caused by aging (P<0.01). The regulatory effects of the Sample-1, 2, and 3 groups were comparable to that of the positive control group (P>0.05), and were significantly better than those of the Sample-4 and 5 groups (P<0.05), indicating that the composition of AFG and FruArg can improve the aging status of penile tissue in aging organisms, and the effect is even better when the mass ratio of AFG to FruArg is 5:1 to 1:3.

### 3) the Effect of the Composition of AFG and FruArg on Erectile Dysfunction Caused by Aging

TABLE 5
The number of penile erections during a set
time in each group of rats in the experiment

| | Average number of erections |
|---|---|
| C | 6.17 ± 1.17 c |
| D | 1.17 ± 0.75a |
| P | 5.00 ± 0.63 c |
| Sample-1 | 5.8 ± 0.72 c |
| Sample-2 | 5.6 ± 0.77 c |
| Sample-3 | 5.85 ± 0.78 c |
| Sample-4 | 3.4 ± 0.47 b |
| Sample-5 | 2.7 ± 0.42 b |

Note:
“a-c” indicates that groups with different letters have significant differences (P < 0.05).

As shown in Table 5, compared with the D-gal model group, the number of penile erections in rats in the AFG and FruArg composition treatment groups significantly increased (P<0.01). The Sample-1, 2, and 3 groups had no significant difference from the positive control group (P>0.05), and were significantly better than the Sample-4 and 5 groups (P<0.05).

In summary, the composition of AFG and FruArg can effectively alleviate sexual dysfunction caused by aging, improve the aging status of penile tissue in aging organisms, and increase the average number of erections. The best effect is achieved when the mass ratio of AFG to FruArg is 5:1 to 1:3.

### Example 2

A method for preparing a red ginseng product capable of alleviating male sexual dysfunction caused by aging was performed following the steps below:
(1) Fresh and insect-free ginseng was selected, the moisture content thereof was adjusted to 65%, and the ginseng was sealed. The moisture content was adjusted specifically by placing the ginseng in a 55° C. oven to decrease the moisture content to 65%. The moisture content was measured using a rapid moisture content tester.
(2) Sealed ginseng was insulated at 55° C. for 48 h, and then the glucose, maltose, and arginine content in the ginseng was measured. By measurement, in the ginseng after insulation, the glucose content increased from 1.30% to 2.84%, the maltose content increased from 2.2% to 10.56%, and the arginine content increased from 3.88% to 4.15%.
(3) Insulated ginseng was unpacked and placed in a 95° C. oven to remove moisture until the moisture content decreased to 30%.
(4) The ginseng from step (3) was sealed again and insulated at 95° C. for 150 min.
(5) The ginseng from step (4) was unpacked and dried at 105° C. until the moisture content was 10% or lower.
(6) The ginseng from step (5) was pulverized, weighed, and packed to obtain the red ginseng product.

By measurement, the sum of the AFG content and FruArg content in the red ginseng product was 78.86 mg/g DW, with an AFG content of 60.69 mg/g DW, a FruArg content of 18.17 mg/g DW, an AFG:FruArg ratio of 3.34:1, a 5-HMF content of 0.0586 mg/g DW, and an AA content of 0.0314 mg/g DW.

### Example 3

A method for preparing a processed Polygonatum sibiricum product capable of alleviating male sexual dysfunction caused by aging was performed following the steps below:
(1) Fresh and insect-free Polygonatum sibiricum was selected, the moisture content thereof was adjusted to 70%, and the Polygonatum sibiricum was sealed. The moisture content was adjusted specifically by placing the Polygonatum sibiricum in a 55° C. oven to decrease the moisture content to 70%. The moisture content was measured using a rapid moisture content tester.
(2) Sealed Polygonatum sibiricum was insulated at 55° C. for 60 h, and then the glucose, maltose, and arginine content in the Polygonatum sibiricum was measured. By measurement, in the Polygonatum sibiricum after insulation, the glucose content increased from 2.50% to 2.77%, the maltose content increased from 2.30% to 3.17%, and the arginine content increased from 1.65% to 1.89%.
(3) Insulated Polygonatum sibiricum was unpacked and placed in a 95° C. oven to remove moisture until the moisture content decreased to 20%.
(4) The Polygonatum sibiricum from step (3) was sealed again and insulated at 100° C. for 30 min.
(5) The Polygonatum sibiricum from step (4) was unpacked and dried in a 100° C. oven until the moisture content was 10% or lower.
(6) The Polygonatum sibiricum from step (5) was weighed and packed to obtain the processed Polygonatum sibiricum product.

By measurement, the sum of the AFG content and FruArg content in the processed Polygonatum sibiricum product was 28.35 mg/g DW, with an AFG content of 16.07 mg/g DW, a FruArg content of 12.28 mg/g DW, an AFG:FruArg ratio of 1.3:1, a 5-HMF content of 0.061 mg/g DW, and an AA content of 0.0349 mg/g DW.

### Example 4

A method for preparing a processed Cistanche deserticola product capable of alleviating male sexual dysfunction caused by aging was performed following the steps below:
(1) Fresh and insect-free Cistanche deserticola was selected, the moisture content thereof was adjusted to 80%, and the Cistanche deserticola was sealed. The moisture content was adjusted specifically by placing the Cistanche deserticola in a 50° C. oven to decrease the moisture content to 80%. The moisture content was measured using a rapid moisture content tester.
(2) Sealed Cistanche deserticola was insulated at 50° C. for 72 h, and then the glucose, maltose, and arginine content in the Cistanche deserticola was measured. By measurement, in the Cistanche deserticola after insulation, the glucose content increased from 3.16% to 4.84%, the maltose content increased from 1.02% to 2.06%, and the arginine content increased from 1.34% to 1.51%.
(3) Insulated Cistanche deserticola was unpacked and placed in a 95° C. oven to remove moisture until the moisture content decreased to 15%.
(4) The Cistanche deserticola from step (3) was sealed again and insulated at 105° C. for 30 min.
(5) The Cistanche deserticola from step (4) was unpacked and dried at 105° C. until the moisture content was 10% or lower.
(6) The Cistanche deserticola from step (5) was pulverized, weighed, and packed to obtain the processed Cistanche deserticola product.

By measurement, the sum of the AFG content and FruArg content in the processed Cistanche deserticola product was 25.77 mg/g DW, with an AFG content of 7.39 mg/g DW, a FruArg content of 18.38 mg/g DW, an AFG:FruArg ratio of 0.4:1, a 5-HMF content of 0.0186 mg/g DW, and an AA content of 0.0155 mg/g DW.

### Example 5

A method for preparing a processed red ginseng and Polygonatum sibiricum product capable of alleviating male sexual dysfunction caused by aging was performed following the steps below:
(1) Fresh and insect-free ginseng and Polygonatum sibiricum were selected respectively, the moisture content thereof was adjusted to 65%, and the ginseng and the Polygonatum sibiricum were sealed. The moisture content was adjusted specifically by placing the ginseng and the Polygonatum sibiricum in a 60° C. oven to decrease the moisture content to 65%. The moisture content was measured using a rapid moisture content tester.
(2) Sealed ginseng and Polygonatum sibiricum were insulated at 60° C. for 72 h respectively, and then the glucose, maltose, and arginine content in the ginseng and the Polygonatum sibiricum was measured. By measurement, in the ginseng after insulation, the glucose content increased from 1.24% to 2.76%, the maltose content increased from 2.56% to 10.08%, and the arginine content increased from 3.84% to 4.29%; and in the Polygonatum sibiricum after insulation, the glucose content increased from 2.50% to 2.85%, the maltose content increased from 2.30% to 3.32%, and the arginine content increased from 1.65% to 1.94%.
(3) Insulated ginseng and Polygonatum sibiricum were unpacked and placed in a 95° C. oven to remove moisture respectively until the moisture content decreased to 25%.
(4) The ginseng and the Polygonatum sibiricum from step (3) were mixed in a mass ratio of 1:1 and pulverized, and sealed again and insulated at 105° C. for 60 min.
(5) The combined material from step (4) was unpacked and dried in a 105° C. oven until the moisture content was 10% or lower.
(6) The material from step (5) was weighed and packed to obtain the processed red ginseng and Polygonatum sibiricum product.

By measurement, the sum of the AFG content and FruArg content in the processed red ginseng and Polygonatum sibiricum product was 55.42 mg/g DW, with an AFG content of 40.46 mg/g DW, a FruArg content of 14.96 mg/g DW, an AFG:FruArg ratio of 2.7:1, a 5-HMF content of 0.0565 mg/g DW, and an AA content of 0.0177 mg/g DW.

### Example 6

A method for preparing a processed red ginseng and Cistanche deserticola product capable of alleviating male sexual dysfunction caused by aging was performed following the steps below:
(1) Fresh and insect-free ginseng and Cistanche deserticola were selected, mixed in a mass ratio of 2:1, and pulverized to obtain a mixed raw material, the moisture content thereof was adjusted to 70%, and the mixed raw material was sealed. The moisture content was adjusted specifically by placing the ginseng and the Cistanche deserticola in a 55° C. oven to decrease the moisture content to 70%. The moisture content was measured using a rapid moisture content tester.
(2) Sealed mixed raw material was insulated at 55° C. for 60 h, and then the glucose, maltose, and arginine content in the mixed raw material was measured. By measurement, in the ginseng and Cistanche deserticola combined material after insulation, the glucose content increased from 2.24% to 3.76% (dry weight), the maltose content increased from 2.24% to 6.78% (dry weight), and the arginine content increased from 2.25% to 2.95% (dry weight).
(3) Insulated mixed raw material was unpacked and placed in a 95° C. oven to remove moisture until the moisture content decreased to 20%.
(4) The mixed raw material from step (3) was sealed again and insulated at 100° C. for 30 min.
(5) The mixed raw material from step (4) was unpacked and dried in a 100° C. oven until the moisture content was 10% or lower.
(6) The mixed raw material from step (5) was weighed and packed to obtain the processed red ginseng and Cistanche deserticola product.

By measurement, the sum of the AFG content and FruArg content in the processed red ginseng and Cistanche deserticola product was 44.22 mg/g DW, with an AFG content of 29.12 mg/g DW, a FruArg content of 15.10 mg/g DW, an AFG:FruArg ratio of 1.92:1, a 5-HMF content of 0.035 mg/g DW, and an AA content of 0.031 mg/g DW.

### Example 7

A method for preparing a composite processed ginseng, Polygonatum sibiricum, and Cistanche deserticola product capable of alleviating male sexual dysfunction caused by aging was performed following the steps below:
(1) Fresh and insect-free ginseng, Polygonatum sibiricum, and Cistanche deserticola were selected, mixed in a mass ratio of 1:1:1, and pulverized to obtain a mixed raw material, the moisture content thereof was adjusted to 70%, and the mixed raw material was sealed. The moisture content was adjusted specifically by placing the ginseng, the Polygonatum sibiricum, and the Cistanche deserticola in a 50° C. oven to decrease the moisture content to 70%. The moisture content was measured using a rapid moisture content tester.
(2) Sealed mixed raw material was insulated at 50° C. for 72 h, and then the glucose, maltose, and arginine content in the mixed raw material was measured. By measurement, in the ginseng, Polygonatum sibiricum, and Cistanche deserticola combined material after insulation, the glucose content increased from 3.54% to 5.66%, the maltose content increased from 3.08% to 5.45%, and the arginine content increased from 2.25% to 2.95%.
(3) The mixed raw material from step (2) was unpacked and placed in a non-sealed environment, dried at high temperature of 90° C. until the moisture content decreased to 20%, and then sealed and steamed at 90° C. for 120 min.
(4) The mixed raw material from step (3) was taken out and dried at 90° C. until the moisture content was 10% or lower.
(5) The mixed raw material from step (4) was weighed, vacuum sealed, and packed to obtain the composite processed ginseng, Polygonatum sibiricum, and Cistanche deserticola product.

By measurement, the sum of the AFG content and FruArg content in the composite processed ginseng, Polygonatum sibiricum, and Cistanche deserticola product was 32.07 mg/g DW, with an AFG content of 15.82 mg/g DW, a FruArg content of 16.25 mg/g DW, an AFG:FruArg ratio of 0.97:1, a 5-HMF content of 0.0198 mg/g DW, and an AA content of 0.0152 mg/g DW.

### Example 8

A method for preparing a processed Polygonatum sibiricum and Cistanche deserticola product capable of alleviating male sexual dysfunction caused by aging was performed following the steps below:
(1) Fresh and insect-free Polygonatum sibiricum and Cistanche deserticola were selected, mixed in a mass ratio of 1:2, and pulverized to obtain a mixed raw material, the moisture content thereof was adjusted to 80%, and the mixed raw material was sealed. The moisture content was adjusted specifically by placing the Polygonatum sibiricum and the Cistanche deserticola in a 50° C. oven to decrease the moisture content to 80%. The moisture content was measured using a rapid moisture content tester.
(2) Sealed mixed raw material was insulated at 50° C. for 72 h, and then the glucose, maltose, and arginine content in the mixed raw material was measured. By measurement, in the Polygonatum sibiricum and Cistanche deserticola combined material after insulation, the glucose content increased from 3.28% to 4.88%, the maltose content increased from 1.09% to 2.49%, and the arginine content increased from 2.06% to 2.35%.
(3) Insulated mixed raw material was unpacked and placed in a 105° C. oven to remove moisture until the moisture content decreased to 20%.
(4) The mixed raw material from step (3) was sealed again and insulated at 105° C. for 60 min.
(5) The mixed raw material from step (4) was unpacked and dried in a 105° C. oven until the moisture content was 10% or lower.
(6) The mixed raw material from step (5) was weighed and packed to obtain the processed Polygonatum sibiricum and Cistanche deserticola product.

By measurement, the sum of the AFG content and FruArg content in the processed Polygonatum sibiricum and Cistanche deserticola product was 26.37 mg/g DW, with an AFG content of 6.60 mg/g DW, a FruArg content of 19.77 mg/g DW, an AFG:FruArg ratio of 0.33:1, a 5-HMF content of 0.0358 mg/g DW, and an AA content of 0.0212 mg/g DW.

### Comparative Example 1

A method for preparing a red ginseng product was performed following the steps below:
(1) Fresh and insect-free ginseng was selected, the moisture content thereof was adjusted to 65%, and the ginseng was sealed. The moisture content was adjusted specifically by placing the ginseng in a 55° C. oven to decrease the moisture content to 65%. The moisture content was measured using a rapid moisture content tester.
(2) Sealed ginseng was insulated at 55° C. for 48 h, and then the glucose, maltose, and arginine content in the ginseng was measured. By measurement, in the ginseng after insulation, the glucose content increased from 1.30% to 2.84%, the maltose content increased from 2.2% to 10.56%, and the arginine content increased from 3.88% to 4.15%.
(3) The ginseng from step (2) was sealed and insulated at 95° C. for 150 min.
(4) The ginseng from step (3) was unpacked and dried at 105° C. until the moisture content was 10% or lower.
(5) The ginseng from step (4) was pulverized, weighed, and packed to obtain the red ginseng product.

By measurement, the sum of the AFG content and FruArg content in the red ginseng product was 41.25 mg/g DW, with an AFG content of 36.09 mg/g DW, a FruArg content of 5.16 mg/g DW, an AFG:FruArg ratio of 6.99:1, a 5-HMF content of 0.0306 mg/g DW, and an AA content of 0.0183 mg/g DW.

### Comparative Example 2

A method for preparing a red ginseng product was performed following the steps in Example 2, except that the decreased moisture content in step (3) was adjusted to 50% to obtain the red ginseng product.

By measurement, the sum of the AFG content and FruArg content in the red ginseng product was 57.23 mg/g DW, with an AFG content of 49.15 mg/g DW, a FruArg content of 8.08 mg/g DW, an AFG:FruArg ratio of 6.08:1, a 5-HMF content of 0.0429 mg/g DW, and an AA content of 0.0215 mg/g DW.

### Comparative Example 3

A method for preparing a red ginseng product was performed following the steps in Example 2, except that the decreased moisture content in step (3) was adjusted to 10% to obtain the red ginseng product.

By measurement, the sum of the AFG content and FruArg content in the red ginseng product was 30.78 mg/g DW, with an AFG content of 27.51 mg/g DW, a FruArg content of 3.27 mg/g DW, an AFG:FruArg ratio of 8.41:1, a 5-HMF content of 0.0136 mg/g DW, and an AA content of 0.0079 mg/g DW.

### Comparative Example 4

A method for preparing a red ginseng product was performed following the steps in Example 2, except that the moisture content in step (1) was adjusted to 90% to obtain the red ginseng product.

By measurement, the sum of the AFG content and FruArg content in the red ginseng product was 69.31 mg/g DW, with an AFG content of 58.57 mg/g DW, a FruArg content of 10.74 mg/g DW, an AFG:FruArg ratio of 5.45:1, a 5-HMF content of 0.0482 mg/g DW, and an AA content of 0.0253 mg/g DW.

### Comparative Example 5

A method for preparing a red ginseng product was performed following the steps in Example 2, except that the moisture content in step (1) was adjusted to 50% to obtain the red ginseng product.

By measurement, the sum of the AFG content and FruArg content in the red ginseng product was 61.81 mg/g DW, with an AFG content of 52.89 mg/g DW, a FruArg content of 8.92 mg/g DW, an AFG:FruArg ratio of 5.93:1, a 5-HMF content of 0.0385 mg/g DW, and an AA content of 0.0183 mg/g DW.

By comparing Comparative Example 1 with Example 2, it can be seen that the sum of the AFG content and FruArg content in the red ginseng product prepared by the technology without step (3) has decreased, and also, the AFG:FruArg ratio does not meet the demand for effectively alleviating male sexual dysfunction caused by aging. In addition, the moisture content which is adjusted to be too high (Comparative Example 2) or too low (Comparative Example 3) in step (3) also leads to a decrease in the AFG and FruArg content, and also, the AFG:FruArg ratio does not meet the demand for effectively alleviating male sexual dysfunction caused by aging.

By comparing Comparative Examples 4 and 5 with Example 2, it can be found that the water adjustment in step (1) is also related to the sum of the AFG content and FruArg content in the obtained red ginseng products, as well as the effectiveness in alleviating male sexual dysfunction. A too high (Comparative Example 4) or too low (Comparative Example 5) moisture content may cause a decrease in the AFG and FruArg content and affect the AFG:FruArg ratio.

In summary, the moisture adjustment in steps (1) and (3) is crucial for increasing the sum of the AFG content and FruArg content in the processed products, as well as making the AFG:FruArg ratio in a range of 5:1 to 1:3. The moisture content is to be controlled at 65-80% in step (1), and at 15-30% in step (3).

### Example 9 Evaluation of Effect of Food Ingredient Rich in AFG and FruArg on Improving Erectile Dysfunction Caused by Aging

Experimental animals: 40 healthy male 8-week-old SPF SD rats.

Experimental grouping: Group C: Blank control group, administered with 0.5% CMC-Na normal saline by gavage; Group D: D-galactose (D-gal) model control group, administered with 0.5% CMC-Na normal saline by gavage; Group P: Tadalafil positive control group, administered with 0.52 mg/kg BW by gavage; Sample-L group: Low-dose sample group, with AFG: FruArg=5:1, the gavage sample is 40 mg/kg BW, calculated as the sum of AFG and FruArg; and Sample-H group: High-dose sample group, with AFG: FruArg=1:3, the gavage sample is 80 mg/kg BW, calculated as the sum of AFG and FruArg.

Except Group C, the other groups received intraperitoneal injections of D-gal at a dose of 150 mg/kg BW daily for 9 consecutive weeks. From the 6th week, intraperitoneal injection and gavage administration were performed simultaneously for 4 consecutive weeks.

The sample used in the Sample-L group is from the red ginseng product prepared in Example 2; and the sample used in the Sample-H group is from the Polygonatum sibiricum-Cistanche deserticola product prepared in Example 8.

### Test Results:

### 1) the Effect of the AFG and FruArg on Aging Body Weight and Penis Weight

TABLE 6
Average body weight and penis weight of rats
in each group during the experimental process

| | C | D | P | Sample-L | Sample-H |
|---|---|---|---|---|---|
| Average | 230.14 ± | 226.57 ± | 232.86 ± | 233.71 ± | 226.43 ± |
| body weight | 10.92a | 9.98a | 10.49a | 12.23a | 8.97a |
| change (g) | | | | | |
| Penis weight | 0.77 ± | 0.77 ± | 0.79 ± | 0.80 ± | 0.78 ± |
| (g) | 0.07a | 0.05a | 0.06a | 0.08a | 0.04a |

Note:
“a” represents the same indicator, and different groups have no significant difference (P > 0.05).

Note: “a” represents the same indicator, and different groups have no significant difference (P>0.05).

As shown in Table 6, the groups have no significant difference in body weight and penis weight (P>0.05), indicating that the red ginseng product and the Polygonatum sibiricum and Cistanche deserticola product have no significant regulatory effect on the body weight and penis weight of rats.

### 2) the Effect of the AFG and FruArg on Aging-Related Marker Genes in the Aging Penis

TABLE 7
Relative expression values of aging-related marker
genes in each experimental group of rats

| | C | D | P | Sample-L | Sample-H |
|---|---|---|---|---|---|
| P53 | 1.00 ± 0.06a | 14.55 ± 1.98d | 2.12 ± 0.47 c | 2.29 ± 0.43c | 1.52 ± 0.34b |
| P21 | 1.00 ± 0.05a | 10.3 ± 1.92d | 1.26 ± 0.22 c | 1.94 ± 0.40 c | 1.44 ± 0.42 b |

Note:
“a-d” indicates that groups with different letters in the same indicator have significant differences (P < 0.05).

As shown in Table 7, compared with the D-galactose (D-gal) model control group, the Sample-L and Sample-H groups of rats showed a significant decrease in the expression of two aging-related genes p53 and p21 in the penile tissue (P<0.01). The rats in the Sample-H group had better regulatory effect on p53 expression than those in the positive control group (P<0.05), the Sample-L group had no inhibitory effect on p53 expression significantly different from the positive control group (P>0.05), and the rats in the Sample-H group had no inhibitory effect on p21 expression significantly different from the positive control group (P>0.05). The results prove that the red ginseng product and the Polygonatum sibiricum-Cistanche deserticola product rich in AFG and FruArg can improve the aging status of penile tissue in aging organisms.

### 3) the Effect of the AFG and FruArg on Erectile Dysfunction Caused by Aging

TABLE 8
The number of penile erections during a set
time in each group of rats in the experiment

| Analyte | C | D | P | Sample-L | Sample-H |
|---|---|---|---|---|---|
| Average | 6.17 ± | 1.17 ± | 5.00 ± | 5.67 ± | 5.83 ± |
| number of | 1.17a | 0.75c | 0.63b | 0.81a | 0.75a |
| erections | | | | | |

Note:
“a-c” indicates that groups with different letters have significant differences (P < 0.05).

As shown in Table 8, compared with the model control group, the number of penile erections in rats in the Sample-L and Sample-H treatment groups significantly increased (P<0.01), and the two experimental groups showed better results than the positive control group (P<0.05).

In summary, both the red ginseng product and the Polygonatum sibiricum-Cistanche deserticola product prepared in the present disclosure are rich in AFG and FruArg in a mass ratio of 5:1 to 1:3, and can effectively alleviate male sexual dysfunction caused by aging.

The examples provided above are not intended to limit the scope covered by the present disclosure, nor are the steps described to limit the execution order. Apparent improvements made to the present disclosure by those skilled in the art based on existing common knowledge also fall within the scope of protection defined in the claims of the present disclosure.

What is claimed is:

1. A method for preparing a processed product with a high arginyl-fructosyl-glucose (AFG) and fructosyl-arginine (FruArg) content and an ability to alleviate male sexual dysfunction caused by aging, the method comprising the following steps:
(1) adjusting a moisture content of raw materials of the processed product to 60%-80% and sealing the raw materials, the raw materials of the processed product being one or more of ginseng, Cistanche deserticola, and Polygonatum sibiricum;
(2) insulating sealed raw materials of the processed product;
(3) taking out insulated raw materials of the processed product and drying the insulated raw materials until a moisture content thereof decreases to 15%-40%;
(4) sealing and insulating the raw materials of the processed product from step (3); and
(5) taking out the raw materials of the processed product from step (4) and drying the raw materials until a moisture content thereof is 10% or lower, to obtain the processed product.

2. The preparation method according to claim 1, wherein the raw materials of the processed product in step (1) comprise intact raw materials, cut raw materials, pulverized raw materials, or pulped raw materials.

3. The preparation method according to claim 1, wherein the ginseng, the Cistanche deserticola, and the Polygonatum sibiricum in step (1) are grown for five years or less.

4. The preparation method according to claim 1, wherein when the raw materials of the processed product are a mixture of the ginseng and the Polygonatum sibiricum, a mass ratio of the ginseng to the Polygonatum sibiricum is (0.5-1.5):1.

5. The preparation method according to claim 1, wherein when the raw materials of the processed product are a mixture of the ginseng and the Cistanche deserticola, a mass ratio of the ginseng to the Cistanche deserticola is (1.5-2.5):1.

6. The preparation method according to claim 1, wherein when the raw materials of the processed product are a mixture of the Polygonatum sibiricum and the Cistanche deserticola, a mass ratio of the Polygonatum sibiricum to the Cistanche deserticola is (0.5-1.5):2.

7. The preparation method according to claim 1, wherein when the raw materials of the processed product are a mixture of the ginseng, the Polygonatum sibiricum, and the Cistanche deserticola, a mass ratio of the ginseng to the Polygonatum sibiricum to the Cistanche deserticola is (0.5-1.5):(0.5-1.5):1.

8. The preparation method according to claim 1, wherein a process of adjusting the moisture content in step (1) is: placing the raw materials of the processed product at 50° C.-60° C. to remove moisture.

9. The preparation method according to claim 1, wherein sealing and insulation in step (2) are performed at 50° C.-60° C. for 48-72 hours.

10. The preparation method according to claim 1, wherein drying in step (3) comprises high-temperature drying, natural air drying, or drying under reduced pressure.

11. The preparation method according to claim 1, wherein the moisture content in step (3) is reduced to 15%-30%.

12. The preparation method according to claim 1, wherein the raw materials of the processed product from step (3) in step (4) are any one of the ginseng, the Cistanche deserticola, and the Polygonatum sibiricum treated in steps (1) to (3).

13. The preparation method according to claim 1, wherein the raw materials of the processed product from step (3) in step (4) are a combination of any two or three of the ginseng, the Cistanche deserticola, and the Polygonatum sibiricum treated separately in steps (1) to (3).

14. The preparation method according to claim 1, wherein the raw materials of the processed product from step (3) in step (4) are a combination of any two or three of the ginseng, the Cistanche deserticola, and the Polygonatum sibiricum treated together in steps (1) to (3).

15. The preparation method according to claim 1, wherein sealing and insulation in step (4) are performed at 90° C.-110° C. for 30-150 minutes.

16. The preparation method according to claim 1, wherein drying in step (5) comprises high-temperature drying, natural air drying, or drying under reduced pressure.

17. The preparation method according to claim 1, wherein the moisture content after drying in step (5) is 8%-10%.

18. A processed product with a high AFG and FruArg content and an ability to alleviate male sexual dysfunction caused by aging prepared by the preparation method according to claim 1, wherein a mass ratio of AFG to FruArg in the processed product is 8:1 to 1:3.

19. The processed product according to claim 18, wherein the mass ratio of AFG to FruArg in the processed product is 5:1 to 1:3.
